(19) United States
(12) Patent Application Publication
(10) Pub. No.: US 2011/0125167 A1
(43) Pub. Date: May 26, 2011

(54) DEVICE FOR TREATING STINGS FROM STINGING ORGANISIMS

(75) Inventor: Per Kirkegaard Hansen, Skanderborg (DK)

(73) Assignee: SKANCARD APS, Horsens (DK)

(21) Appl. No.: 12/674,567

(22) PCT Filed: Aug. 22, 2008
(86) PCT No.: PCT/DK08/50211

§ 371 Date: Aug. 25, 2010

(30) Foreign Application Priority Data
Aug. 22, 2007 (DK) PA 2007 01201

(51) Int. Cl. A61F 17/00; A61B 17/50

(52) U.S. Cl. 606/131; 604/289

(57) ABSTRACT

A device is provided, the device being adapted for keeping in a wallet, a pocket, a bag, or similar place portable to a person, where the device is designed substantially as a credit card in size with a length of between 75 mm and 85 mm, possibly approximately 83 mm, a width of between 45 mm and 55 mm, possibly approximately 54 mm, and a thickness of maximum 5 mm, where card is preferably made of a relatively stiff material, such as cardboard, metal, wood or plastic, the card having at least one edge being sharpened in relation to a remaining part of the card, where the card has at least one recess or location for holding at least one agent for applying on the skin of a person or an animal.

### TECHNICAL FIELD

The invention relates to treating a sting of stinging organisms after having been stung. The invention relates to providing agents for inhibiting or alleviating the sourness and illness of having been stung, and in relation to some very dangerous species of coelenterates, the invention relates to agents for surviving after having been stung.

### BACKGROUND

Jellyfish are part of an aquatic invertebrate phylum known as coelenterates. This group of creatures is divided into four different classes: hydrozoa (fire coral), scyphozoa (sea nettle), cubozoa (box jellyfish), and anthozoa (sea anemone). Coelenterates are responsible for more envenomations or injections of venom than any other marine phylum.

Jellyfish are marine invertebrates having a bell-shaped, jelly-like substance enclosing its internal structure.

Most species of jellyfish have tentacles with stinging cells. The stinging cells may stun or kill other ocean life. Most jellyfish use stinging to either stunning prey before catching the prey or to defend themselves. Most jellyfish are not very dangerous to humans, but a few are highly toxic such as the box jelly fish in the Pacific Ocean along the Australian east coast and along the costs of Hawaii.

It is known to use different liquids for relieving the pain caused by the sting, subsequently to remove the stingers from the skin by scraping off the stingers from the skin, and finally to use antihistamines for soothing any final skin irritation.

Bees are flying insects closely related to wasps and ants. There are nearly 20,000 known species of bee, in nine recognized families. They are ubiquitous, present in every habitat on the planet that contains insect-pollinated flowering plants.

A bee that is away from the hive foraging for nectar or pollen will rarely sting. Except when stepped on or roughly handled. However, bees will actively seek out and sting when they perceive the hive to be threatened, often being alerted to this by the release of attack pheromones from other bees. The sting of a bee is barbed so that it lodges in the victim's skin, tearing loose from the bee's abdomen and leading to its death in minutes.

The main component of bee venom (apitoxin) responsible for pain in vertebrates is the toxin melittin, but histamine and other biogenic amines also contribute to pain and especially itching. Histamine is also released from body cells because of the reaction to the sting in allergic persons.

Apitoxin is acidic, and because of that it is often recommended to neutralize the venom. However, neutralizing a sting is unlikely to be effective as the venom is injected under the skin and deep into the tissues, where a topically applied alkali is unable to reach. Medications such as antipruritics, known as anti-itch drugs, are also used in locally treating an insect sting. When being stung by a bee, the immediate action is to scrape out the sting to avoid further venom to enter the body.

All the liquids and remedies for the above mentioned procedures are not always at hand where needed, namely at the beach or in the forest, and all liquids are even not always at hand at home.

US2007/0280926 A1 describes a sting apparatus and method of use. The apparatus is a portable kit that can be easily used to immediately treat jelly fish stings or stings from other stinging organisms such as bees, wasps, hornets, etc. The kit includes a solution including a proteinase toxin neutralizer to neutralize jelly fish stinger toxin proteins and tools coated with proteolytic agents to attract, attach and further neutralize jelly fish stingers.

### BRIEF SUMMARY

In order to ensure fast, correct and satisfactory treatment of persons having been stung by stinging organisms such as coelenterates, stinging nettles, or bees, it would be an advantage to have the agents and remedies for treating the stings combined in a single device of relatively small size to carry around at all times. The disclosure seeks to overcome the disadvantages of not having the agents and remedies for the above mentioned procedures at hand where needed, namely at the beach or in the forest, and all agents are even not always at hand at home.

The treatment could also be used for animals such as pets, zoo animals or wild animals that may have become stung by stinging organisms such as coelenterates, stinging nettles, or bees. The disclosure also seeks to decrease the risk of being ill or even die from being stung, and to enhance people to take necessary precautions.

A device is provided adapted for keeping in a wallet, a pocket, a bag, or similar place portable to a person, said device being designed as a credit card in size with a length of approximately 85 mm, a width of approximately 54 mm, and a thickness of maximum 5 mm, and said card being made of a relatively stiff material, such as cardboard, wood, metal or plastic, said card having at least one edge being sharpened in relation to the remaining part of the card, and said card having at least one recess or location for holding at least one agent for applying on the skin of a person or an animal.

In another embodiment, a device is provided for use in treating sting by a stinging organism, said treatment being performed by removing stingers from the skin, and said treatment also being performed by applying at least one agent on the skin of a person or an animal for relieving pain and/or for soothing the skin, said device being adapted for keeping in a wallet, a pocket, a bag, or similar place portable to a person, said device being designed substantially as a credit card in size with a length of between 75 mm and 85 mm, possibly approximately 83 mm, a width of between 45 mm and 55 mm, possibly approximately 54 mm, and a thickness of maximum 5 mm, and said card preferably being made of a relatively stiff material, such as cardboard, metal, wood or plastic, said card having at least one edge being sharpened in relation to a remaining part of the card, and said card having at least one recess or location for holding at least one agent for applying on the skin of a person or an animal.

As mentioned, the device according to the invention has a size corresponding to a credit card, i.e. with a length of about 85 mm, a width of about 54 mm and a total thickness less than 5 mm, preferably less than 2 mm.

In another embodiment, the device according to the invention has a design as a credit card in size with a length of between 80 mm and 90 mm, a width of between 49 mm and 59 mm, and a thickness of between 0.25 mm and 4.00 mm.

In yet another embodiment, the device according to the invention is designed substantially as a credit card in size, has a length of between 80 mm and 85 mm, a width of between 45 mm and 55 mm, and a thickness of between 0.25 mm and 3 mm.

Alternatively, the device according to the invention is designed substantially as a credit card in size, has a length of between 80 mm and 85 mm, a width of between 50 mm and 55 mm, and a thickness of between 0.25 mm and 3 mm.

In one embodiment, a device according to the invention where said edge being sharpened in relation to a remaining part of the card has a length of between 0.25 mm and 10 mm, and an angle of inclination in relation to an extension of a plane of the card of between 15 degrees and 75 degrees.

In another embodiment, a device according to the invention where said edge being sharpened in relation to a remaining part of the card has a length of between 2 mm and 6 mm, and an angle of inclination in relation to an extension of a plane of the card of between 15 degrees and 45 degrees.

When being stung by a bee, the immediate action is to scrape out the sting to avoid further venom to enter the body.

In one embodiment, a device according to the invention is made so that the card is provided with a slit adapted for use in releasing a bee sting, said slit having an orifice at one edge of the device, preferably having an orifice at one of the at least one sharpened edge of the device.

In another embodiment, a device according to the invention is made so that the card is provided with a slit adapted for use in releasing a bee sting, said slit having an orifice at a corner of the device, preferably having an orifice at a corner positioned along the at least one sharpened edge of the device.

In yet another embodiment, a device according to the invention is made so that the slit adapted for use in releasing a bee sting has a tapering configuration with a wide end of the tapering configuration constituting the orifice and with the narrow end of the tapering configuration being opposite the orifice of the slit.

The immediate treatment of the sting involves relieving the pain and removing the stingers. Regarding stings from coelenterates, prior to removing the stingers, an immediate treatment of the coelenterates' stingers may be performed to inhibit further venom discharge. A subsequent treatment of the sting involves soothing the skin for long-term reducing of pain and itch. Removing the stingers is very important; relieving the pain and itch may be done, but need not be done. Soothing the skin may be done, but need not be done.

The device according to the invention may be designed so that a folded first tissue containing a pain relieving agent has a length and a width of about 44 mm×44 mm, and that the device has a second tissue having a length and a width of about 24×44 mm, said second tissue containing an antipruritic agent or a skin soothing agent or a stinger neutralization agent. In another embodiment, the device according to the invention may be designed so that a folded first tissue containing a stinger neutralization agent has a length and a width of about 44 mm×44 mm, and that the device has a second tissue having a length and a width of about 24×44 mm, said second tissue containing an antipruritic agent or a skin soothing agent or a pain relieving agent.

Alternatively, the folded first tissue may have a length and a width of about 56×44 mm, whereas the second folded tissue may correspondingly have a length and a width of about 12×44 mm, or vice versa, so that the first tissue may be larger than second tissue, or vice versa.

Alternatively, the device according to the invention may be designed so that that a folded first tissue containing a pain relieving agent has a length and a width of about 44 mm×44 mm, and that the device has a second and a third tissue both having a length and a width of about 12×44 mm, said second tissue containing an antipruritic agent or a skin soothing agent, said third tissue containing an antipruritic agent or a skin soothing agent.

Alternatively, the device according to the invention may be designed so that that a folded first tissue containing a stinger neutralization agent has a length and a width of about 44 mm×44 mm, and that the device has a second and a third tissue both having a length and a width of about 12×44 mm, said second tissue containing an antipruritic agent or a skin soothing agent or a pain relieving agent, said third tissue containing an antipruritic agent or a skin soothing agent or a pain relieving agent. Alternatively to having folded tissues with pain relieving and/or skin soothing and/or antipruritic and/or stinger neutralization agents, the card may have recesses or just have designated locations for holding a paste or a flat solid brick of pain relieving agent, and/or antipruritic agent, and/or stinger neutralization agent, and/or skin soothing agent.

The various forms of administration may solve different problems, e.g. if a victim has a large area affected by stingers a large amount or volume of agent might be necessary. This could be solved by the card holding a solid brick of concentrated agent for dilution in water.

Alternatively to having both pain relieving agent and skin soothing agent, the card may have only pain relieving agent, provided on a tissue or as a paste or a solid brick or as foil-wrapped liquid, or the card may have only skin soothing agent, provided on a tissue or as a paste or a solid brick.

Alternatively to having several agents, the card may have only one agent, provided on a tissue or as foil-wrapped liquid or as a paste or a solid brick.

In still alternative embodiments, more than one recess or two recesses or locations may be provided. The device is thus capable of holding more than one or two agents, or the device may hold more amounts of the one or of the two agents, possibly for different doses of one or both agents being applied within intervals during treatment.

Preferably, the device according to the invention is designed so that any tissue holding either the pain relieving agent or the skin soothing agent has a size which is considerably greater that the size of reservoir or the location where the tissue is intended for being positioned on the surface of the card.

Preferably, the device according to the invention is designed so that any tissue holding any type of the before mentioned agents has a size which is considerably greater that the size of recess or the location where the tissue is intended for being positioned on the surface of the card. The tissue is then folded in order for the larger sized tissue to fit the at least one recess or location of the card.

In one embodiment, the device according to the invention comprises at least one agent being, said agent being provided on a tissue or as foil-wrapped liquid or as a paste or a solid brick and being located in the at least one recess or location of the card. Depending on the kind of agent and depending on the need of the agent for immediate use, different means of properties, i.e. impregnated in a tissue, applicable as a paste, or to be dissolved as a solid brick in liquid may be used for the card to hold the agent during production, packaging and storing of the device and during subsequent carrying along of the device by the end user.

In another embodiment, the device according to the invention comprises at least two different agents, said agents being provided on a tissue or as foil-wrapped liquid or as a paste or a solid brick and being located in the at least one recess of the card, possibly being located in two different recesses of the card.

Providing different agents may be useful, either for providing the possibility of soothing pain and/or for curing immediate skin disorder of persons being stung by one or another different organism. Providing different agents may also be useful for providing the possibility of soothing pain and curing immediate skin disorder or other differentiated treatment of person being stung by one organism. The different agents may in one embodiment be provided in two different recesses, said recesses resulting in the overall thickness of the card being minimised.

In yet another embodiment, a device according to the invention comprises at least two different agents, said agents being provided on a tissue or as foil-wrapped liquid or as a paste or a solid brick and being located in the at least one location of the card, possibly being located in two different locations of the card.

Providing different agents may be useful, either for providing the possibility of soothing pain and/or for curing immediate skin disorder of persons being stung by one or another different organism. Providing different agents may also be useful for providing the possibility of soothing pain and curing immediate skin disorder or other differentiated treatment of person being stung by one organism. The different agents may in one embodiment be provided in two different locations, said location resulting in the overall stiffness of the card being maximised

A card may be made more stiff only having locations and not recesses, such card only having locations not having a smaller thickness, and such card only having locations not having a reduced stiffness, where recesses would be located.

Alternatively, a device according to the invention comprises at least two different agents being provided on a tissue or as foil-wrapped liquid or as a paste or a solid brick and each of said at least two different agents being covered by means of differently coloured films, or being of different colour or being provided with or made with other marking for facilitating to discern between the at least two different agents of the device. By being able to differentiate between the different agent, correct and fast treatment of the person to be stung is ensured.

In one embodiment the device according to the invention is intended for being used in treating a sting by a member of the coelenterates, and said at least one recess or location holds at least antivenin as agent for applying on the skin of a person or an animal.

In another embodiment, the device according to the invention is intended for being used in treating a sting by a jellyfish, and said at least one recess or location holds at least one of the following agents: Diluted acetic acid, ammonia, or citrus juice for medical treatment of the person having been stung.

In yet another embodiment, the device according to the invention is intended for being used in treating a sting by a sea nettle, and where said at least one recess or location holds at least baking soda for medical treatment of the person having been stung.

Sea anemones and some kinds of coral also contain nematocysts, and stings from anemones or coral are similar to a jellyfish sting. Sea anemones are small creatures that attach themselves to rocks, coral, or even the backs of crabs. They use their flower-like tentacles to catch small marine animals, which are paralyzed by the anemone's venom.

In a further embodiment, the device according to the invention is intended for being used in treating a sting by a sea anemone, and said at least one recess or location holds at least white vinegar for medical treatment of the person having been stung.

In yet a further embodiment, the device according to the invention is intended for being used in treating a sting by a bee, and said at least one recess or location holds at least diphenhydramine for medical treatment of the person having been stung.

In still a further embodiment, the device according to the invention is intended for being used in treating a sting by a stinging nettle, and said at least one recess or location holds at least aluminium acetate for medical treatment of the person having been stung.

The different agents mentioned above in relation to the different organisms are agents which are well-suited for immediate treatment of a person being stung. Other agents than the ones mentioned will also be possible to provide within the general common knowledge of the person skilled in the art. However, it is preferred that the one or more agents provided are suited for immediate treatment and not are intended for long term treatment only, e.g. antibiotics. Immediate treatment is meant as treatment at site of the person being stung, or within a period of maximum 12 hours after the person have been stung.

In a preferred embodiment, the device according to the invention has at least one edge being sharpened in relation to a remaining part of the card has a length of between 3 mm and 4 mm, and an angle of inclination 10 in relation to an extension of a plane of the card of between 25 degrees and 30 degrees. By providing the at least one edge with a sharpening as mentioned, a proper but still not painful scraping is obtained of the skin of the person being treated.

With the purpose of facilitating to discern between the individual parts of the device, the device may suitably be designed so that the different agents are covered by means of films with mutually different colour or other marking. Suitably, the device is further designed so that the cover films are provided with a corner or tongue section, which is not connected with the device, and which is intended for use in pulling off the cover films.

The device according to the invention is made of a relatively stiff material, e.g. cardboard or metal or wood or plastic for ensuring a proper scraping function of the card. The choice of material depends on the intended use of the card, i.e. which organism a person being stung is intended for being treated for, and depends on the maximum price and/or the intended size of the device, especially the intended thickness of the device, and especially the intended thickness of the card, apart from the agents, of the device.

### BRIEF DESCRIPTION OF THE DRAWINGS

FIG. 1 shows a card having a plurality of recesses;

FIG. 2 shows another card with recesses;

FIG. 3 shows a card having slit feature; and

FIGS. 4A-D show side views and enlarged partial cross-sections of a card.

### DETAILED DESCRIPTION

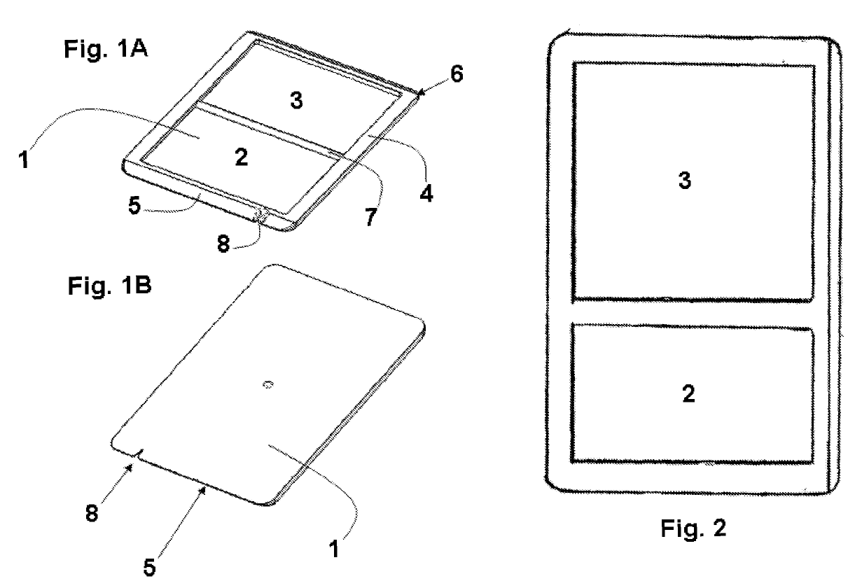

FIG. 1 shows a card 1 having a selected dimension corresponding to the size of a credit card. The card thus have a length of approximately 85 mm, preferably a length of 83 mm, a width of approximately 54 mm and a thickness of maximum 5 mm, preferably a thickness of maximum 3 mm such as approximately 2 mm. In the embodiment shown, all corners 6 of the card are rounded. Alternatively, all or some of the corners 6 of the card may be pointed or rounded with another radius than the one shown.

The card is provided with a first recess 2 and a second recess 3 provided in the surface 4 of the card and divided by a division bulge 7. The recesses 2,3 are intended for holding different agents for immediate relieving pain and for subsequent soothing of the skin. The recesses 2, 3 are shown to be of the same size. The recesses may however be of different size as seen in FIG. 2, or the first recess may be larger than the second recess. Depending on the agent used for relieving the pain and the agent used for soothing the skin, possibly the one agent needs to be applied in a larger amount than the other agent, or vice versa.

Alternatively, one or both of the recesses are omitted, and the areas of the card where the recesses are illustrated may be used as selected locations of the surface 4 of the card for the agents for relieving the pain and for soothing the skin, however, without recesses as such provided in the surface of the card for holding the agents. The agents may be provided contained in a tissue, as a foil-wrapped liquid, or applied as a paste or provided as a small solid brick.

The first recess 2 or location of the card is intended for holding an agent for neutralizing the stingers and relieving the pain as part of an immediate treatment after having been stung by a stinging organism such as a jellyfish. The agent for neutralizing the stingers and relieving the pain may be provided as a solid brick of concentrated agent for dilution in water provided in the recess 2 or he location, or as a foil-wrapped liquid provided in the recess 2 or location, or the agent for neutralizing the stinger and relieving the pain may be provided as a paste filled into the recess 2, or contained in a tissue folded to fit the recess 2.

The agent for neutralizing the stinger and relieving the pain as part of an immediate treatment may be one or more of any of the following substances, either provided as one of the substances only or provided as a selected mixture of more substances: A 5% to 25% acetic acid solution such as vinegar, ammonia, or citrus juice, meat tenderizer, sodium bicarbonate such as baking soda, diphenhydramine, aluminium acetate, a deactivating solution or sea water.

The second recess 3 or location of the card is holding an agent for soothing the skin after having performed the immediate treatment including neutralizing the stinger and relieving the pain. The agent for soothing the skin may be provided on a tissue folded to fit the recess or the location, or as a foil-wrapped liquid provided in the recess 2 or he location, or the agent for soothing the skin may be provided as a paste filled into the recess or as a small brick.

The agent for soothing the skin as part of a subsequent treatment may be one or more of any of the following substances, either provided as one of the substances only or provided as a selected mixture of more substances: antihistamines containing diphen-hydramine such as Benadryl™, hydrocortisone cream, antivenom comprising antibodies against the venom's active molecule, any anaesthetic agent or Papaine enzyme.

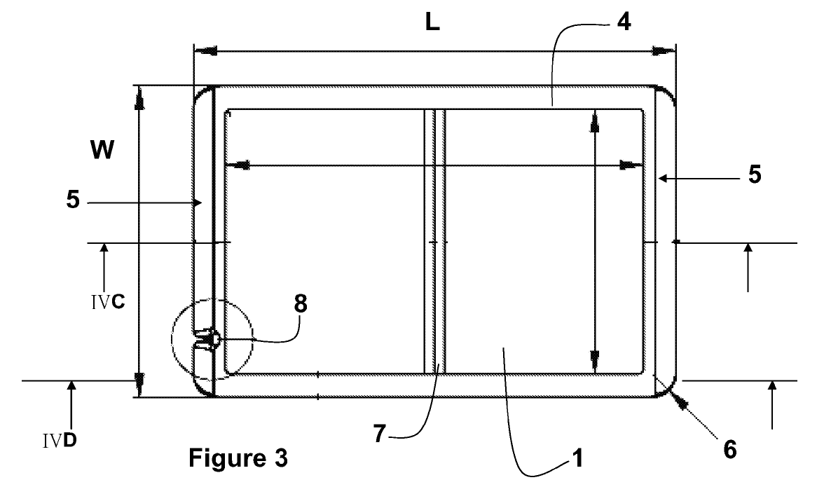

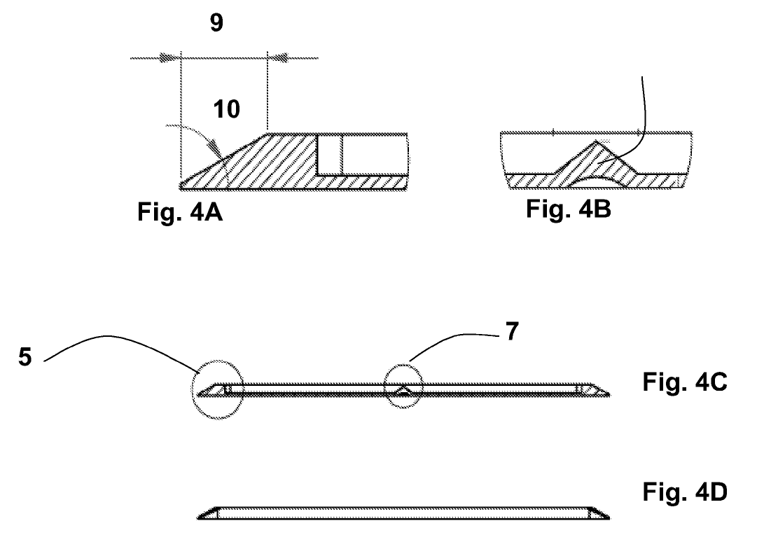

In another preferred embodiment, a device according to the invention is made so that the card is provided with a slit 8 (see FIG. 3) adapted for use in releasing a bee sting, said slit having a tapering configuration with a wide end of the tapering configuration constituting the orifice and with the narrow end of the tapering configuration being opposite the orifice of the slit. The card is preferably provided with one or two sharpened edges 5 along the length of the card and/or along the width of the card. Alternatively the card is provided with three or four sharpened edges along the length of the card and along the width of the card. In the embodiments shown in FIG. 1, the card is provided with two sharp edges 5 along the width of the card. In the embodiments shown in FIG. 2, the card is only provided with one sharp edge 5 along the length of the card.

The one or more sharpened edges are provided by the provision of the one or both edges having a smaller thickness than the overall and/or maximum thickness of the card, such as the thickness immediate outside the edges. One or two or even more sharpened edges make the card more suited for scraping off the stingers from the skin The one or more sharpened edges may be passed along the skin with the sharpened edge leading, so that the stingers are scraped up onto the card and off the skin.

The one or more sharpened edges, if provided on the card, have a thickness of less than 75% of the overall thickness of the card, preferably a thickness of less than 50% of the overall thickness of the card. The sharper the one or more edges are, the better the one or more edges are for scraping off the stingers. It is important that the stingers are scraped off by the edge “shovelling” the stingers by means of the card being pushed along the skin with the sharpened edge along the skin as the leading edge of the card.

Different edges of the card may have different sharpness. This may give the advantage of one edge being for scraping on the skin of a child having a more sensitive skin and another edge being for scraping on the skin of an older person having a less sensitive skin towards scraping of the card along the skin. Also, it may have the advantage of being able of scraping with different sharpened edges more than once along the skin.

In the above, the invention is describes with reference to the accompanying drawing and with reference to specific embodiments. The person skilled in art may come up with other embodiments which all lie within the scope of protection as defined by the claims.

1. A device for use in treating sting by a stinging organism, said treatment being performed by removing stingers from the skin and by applying at least one agent on the skin of a person or an animal for relieving pain and/or for soothing the skin, said device being adapted for keeping in a wallet, a pocket, a bag, or similar place portable to a person, said device being designed substantially as a credit card in size with a length of between 75 mm and 85 mm, a width of between 45 mm and 55 mm, and a thickness of a maximum of 5 mm, and said card preferably being made of a relatively stiff material, said card having at least one edge being sharpened in relation to a remaining part of the card, and said card having at least one recess or location for holding at least one agent for applying on the skin of a person or an animal.

2. A device for use according to claim 1, where the device is intended for being used in treating a sting by a member of the coelenterates, and where said at least one recess or location holds at least antivenin as agent for applying on the skin of a person or an animal.

3. A device for use according to claim 1, where the device is intended for being used in treating a sting by a jellyfish, and where said at least one recess or location holds at least one of the following agents: Diluted acetic acid, ammonia, or citrus juice for medical treatment of the person having been stung.

4. A device for use according to claim 1, where the device is intended for being used in treating a sting by a sea nettle, and where said at least one recess or location holds at least baking soda for medical treatment of the person having been stung.

5. A device for use according to claim 1, where the device is intended for being used in treating a sting by a sea anemone, and where said at least one recess or location holds at least white vinegar for medical treatment of the person having been stung.

6. A device for use according to claim 1, where the device is intended for being used in treating a sting by a bee, and where said at least one recess or location holds at least diphenhydramine for medical treatment of the person having been stung.

7. A device for use according to claim 1, where the device is intended for being used in treating a sting by a stinging nettle, and where said at least one recess or location holds at least aluminium acetate for medical treatment of the person having been stung.

8. A device according to claim 1, where said device being designed substantially as a credit card in size has a length of between 80 and 85 mm, a width of between 45 and 55 mm, and a thickness of between 0.25 and 3 mm.

9. A device according to claim 1, where said device being designed as a credit card in size with a length of between 80-85 mm, a width of between 50-55 mm, and a thickness of between 0.25 and 3 mm.

10. A device according to claim 1, where said edge being sharpened in relation to a remaining part of the card has a length of between 0.25-10 mm, and an angle of inclination in relation to an extension of a plane of the card of between 15 and 75 degrees.

11. A device according to claim 9, where said edge being sharpened in relation to a remaining part of the card has a length of between 2 and 6 mm, and an angle of inclination in relation to a planar extension of the card of between 15 and 45 degrees.

12. A device according to claim 1, wherein said card is provided with a slit adapted for use in releasing a bee sting, said slit having an orifice at one edge of the device.

13. A device according to claim 1, wherein said card is provided with a slit adapted for use in releasing a bee sting, said slit having an orifice at a corner of the device.

14. A device according to claim 12, wherein said slit adapted for use in releasing a bee sting has a tapering configuration with a wide end of the tapering configuration constituting the orifice and with the narrow end of the tapering configuration being opposite the orifice of the slit.

15. A device according to claim 1, where the device comprises at least one agent being provided on a tissue or as foil-wrapped liquid or as a paste or a solid brick and being located in the at least one recess or location of the card.

16. A device according to claim 1, where the device comprises at least two different agents being provided on a tissue or as foil-wrapped liquid or as a paste or a solid brick and being located in the at least one recess of the card or being located in two different recesses of the card.

17. A device according to claim 1, where the device comprises at least two different agents being provided on a tissue or as foil-wrapped liquid or as a paste or a solid brick and being located in the at least one location of the card or in two different locations of the card.

18. A device according to claim 1-17, where the device comprises at least two different agents being provided on a tissue or as foil-wrapped liquid or as a paste or a solid brick, and each of said at least two different agents being covered by means of differently coloured films, or being of different color or being provided with or made with other marking for facilitating to discern between the at least two agents of the device.

19. A device according to claim 13, wherein said slit adapted for use in releasing a bee sting has a tapering configuration with a wide end of the tapering configuration constituting the orifice and with the narrow end of the tapering configuration being opposite the orifice of the slit.
